(19) United States
(12) Patent Application Publication
(10) Pub. No.: US 2022/0087654 A1
(43) Pub. Date: Mar. 24, 2022

(54) ULTRASOUND DIAGNOSIS APPARATUS, IMAGING METHOD, AND COMPUTER PROGRAM PRODUCT

(71) Applicant: CANON MEDICAL SYSTEMS CORPORATION, Otawara-shi (JP)

(72) Inventor: Yoshitaka MINE, Nasushiobara (JP)

(73) Assignee: CANON MEDICAL SYSTEMS CORPORATION, Otawara-shi (JP)

(21) Appl. No.: 17/478,217

(22) Filed: Sep. 17, 2021

(30) Foreign Application Priority Data
Sep. 23, 2020 (JP) 2020-158683

(51) Int. Cl. A61B 8/00; A61B 8/08

(52) U.S. Cl. CPC A61B 8/543; A61B 8/483; A61B 8/4218

(57) ABSTRACT

An ultrasound diagnosis apparatus according to an embodiment includes a robot arm and processing circuitry. The robot arm is capable of moving and rotating an ultrasound probe. The processing circuitry is configured to three-dimensionally perform a scan by using the robot arm. Further, the processing circuitry is configured to evaluate ultrasound data obtained from the scan so as to judge whether or not it is necessary to perform a re-scan using the robot arm.

### CROSS-REFERENCE TO RELATED APPLICATIONS

This application is based upon and claims the benefit of priority from Japanese Patent Application No. 2020-158683, filed on Sep. 23, 2020; the entire contents of which are incorporated herein by reference.

### FIELD

Embodiments described herein relate generally to an ultrasound diagnosis apparatus, an imaging method, and a computer program product.

### BACKGROUND

In recent years, an ultrasound diagnosis apparatus equipped with a robot arm has been proposed. The ultrasound diagnosis apparatus is configured to perform an ultrasound scan by having an ultrasound probe gripped by the robot arm and causing the ultrasound probe to abut against the body surface of an examined subject by using the robot arm. Thus, an attempt is made to obtain images of constant quality without depending on examination manipulations of the operators.

### BRIEF DESCRIPTION OF THE DRAWINGS

FIG. 1 is a diagram illustrating an exemplary configuration of an ultrasound diagnosis apparatus according to an embodiment;

FIG. 2 is a flowchart illustrating a processing procedure performed by the ultrasound diagnosis apparatus according to the embodiment;

FIG. 3 is a drawing for explaining coordinate systems according to the embodiment;

FIG. 4 is a drawing for explaining association between skeleton information and a patient coordinate system according to the embodiment;

FIG. 5 is a drawing for explaining setting an initial position according to the embodiment;

FIG. 6 is a drawing for explaining a motion condition table according to the embodiment;

FIG. 7 is a drawing for explaining processes performed by an evaluating function according to the embodiment; and

FIG. 8 is another drawing for explaining the processes performed by the evaluating function according to the embodiment.

### DETAILED DESCRIPTION

An ultrasound diagnosis apparatus according to an embodiment includes a robot arm and processing circuitry. The robot arm is capable of moving and rotating an ultrasound probe. The processing circuitry is configured to three-dimensionally perform a scan by using the robot arm. Further, the processing circuitry is configured to evaluate ultrasound data obtained from the scan so as to judge whether or not it is necessary to perform a re-scan using the robot arm.

Exemplary embodiments of an ultrasound diagnosis apparatus, an imaging method, and a computer program product will be explained below, with reference to the accompanying drawings. Further, possible embodiments are not limited to the embodiments described below. Further, the description of each of the embodiments is, in principle, similarly applicable to any other embodiment.

### Embodiments

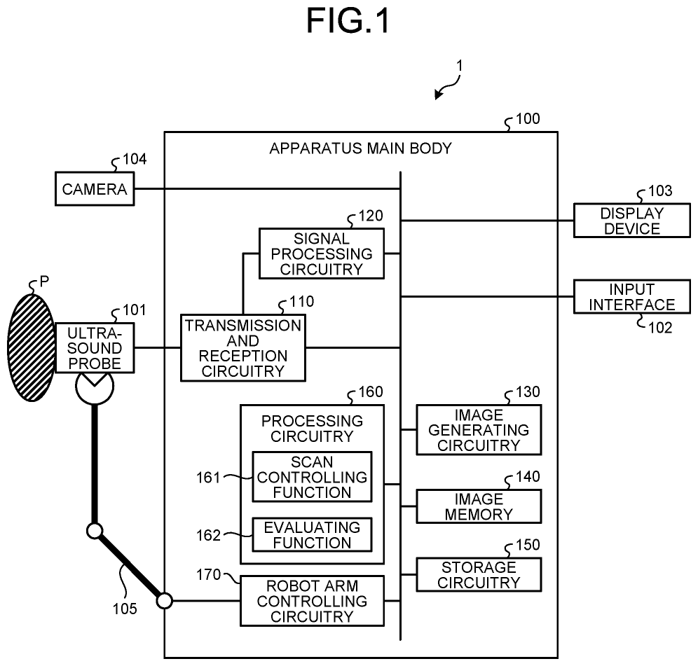

An exemplary configuration of an ultrasound diagnosis apparatus 1 according to an embodiment will be explained, with reference to FIG. 1. FIG. 1 is a diagram illustrating the exemplary configuration of the ultrasound diagnosis apparatus 1 according to the embodiment. As illustrated in FIG. 1, the ultrasound diagnosis apparatus 1 according to the embodiment includes an apparatus main body 100, an ultrasound probe 101, an input interface 102, a display device 103, a camera 104, and a robot arm 105. The ultrasound probe 101, the input interface 102, and the display device 103 are connected to the apparatus main body 100. An examined subject (hereinafter, “patient”) P is not included in the configuration of the ultrasound diagnosis apparatus 1.

The ultrasound probe 101 includes a plurality of transducer elements (e.g., piezoelectric transducer elements). Each of the plurality of transducer elements is configured to generate an ultrasound wave on the basis of a drive signal supplied thereto from transmission and reception circuitry 110 (explained later) included in the apparatus main body 100. Further, each of the plurality of transducer elements included in the ultrasound probe 101 is configured to receive a reflected wave arriving from the patient P and to convert the received reflected wave into an electrical signal. Further, the ultrasound probe 101 includes a matching layer provided for the transducer elements, a backing member that prevents ultrasound waves from propagating rearward from the transducer elements, and the like.

When an ultrasound wave is transmitted from the ultrasound probe 101 to the patient P, the transmitted ultrasound wave is repeatedly reflected on a surface of discontinuity of acoustic impedances at a tissue in the body of the patient P. The reflected ultrasound wave is received as a reflected-wave signal (an echo signal) by the plurality of transducer elements included in the ultrasound probe 101. The amplitude of the received reflected-wave signal is dependent on the difference between the acoustic impedances on the surface of discontinuity on which the ultrasound wave is reflected. When a transmitted ultrasound pulse is reflected on the surface of a moving blood flow, a cardiac wall, or the like, the reflected-wave signal is, due to the Doppler effect, subject to a frequency shift, depending on a velocity component of the moving members with respect to the ultrasound wave transmission direction.

The embodiment is applicable to any of the following situations: the ultrasound probe 101 illustrated in FIG. 1 is a one-dimensional ultrasound probe in which a plurality of piezoelectric transducer elements are arranged in a row; the ultrasound probe 101 is a one-dimensional ultrasound probe in which a plurality of piezoelectric transducer elements arranged in a row are mechanically swung; and the ultrasound probe 101 is a two-dimensional ultrasound probe in which a plurality of piezoelectric transducer elements are two dimensionally arranged in a matric formation.

The input interface 102 includes a mouse, a keyboard, a button, a panel switch, a touch command screen, a foot switch, a trackball, a joystick, and/or the like and is configured to receive various types of setting requests from an operator of the ultrasound diagnosis apparatus 1 and to transfer the received various types of setting requests to the apparatus main body 100.

The display device 103 is configured to display a Graphical User Interface (GUI) used by the operator of the ultrasound diagnosis apparatus 1 to input the various types of setting requests via the input interface 102 and is configured to display ultrasound image data or the like generated by the apparatus main body 100.

The camera 104 is a device configured to image the patient P and the ultrasound probe 101. For example, a scan controlling function 161 (explained later) is configured to obtain position information of the patient P and the ultrasound probe 101, by performing any of various types of image recognition processes on image data (hereinafter, “camera image data”) taken by the camera 104. For example, the scan controlling function 161 is configured to obtain skeleton information of the patient P by performing a skeleton recognition process on the camera image data. The skeleton information is information indicating the positions of a plurality of representative joints of the patient P and the positions of representative bones connecting the joints. Further, the scan controlling function 161 is configured to obtain three-dimensional position information indicating the position and the orientation of the ultrasound probe 101, by performing an image recognition process such as pattern matching on the camera image data.

Alternatively, it is also possible to estimate the position information of the ultrasound probe 101 from position information of the robot arm 105, by using a positional relationship between the ultrasound probe 101 and the robot arm 105. It is possible to obtain the position information of the robot arm 105, by performing an image recognition process on image data taken by the camera described above or making an estimate (through a calculation) from the lengths and rotation angles of arms structuring the robot arm 105.

Further, in the present embodiment, the example provided with the camera 104 was explained; however, possible embodiments are not limited to this example. For instance, in place of the camera 104, it is possible to select and adopt, as appropriate, any of publicly-known techniques for obtaining the position information of the patient P and the ultrasound probe 101, such as those using an infrared sensor, a magnetic sensor, or a sensor system combining various types of sensors together. Further, it is also acceptable to adopt a sensor system using two or more sensors of mutually the same type in combination (e.g., using two or more cameras 104 in combination). The larger the number of sensors or the number of types of sensors used in combination is, the higher will be the precision level of the detection, because blind angles of the sensors can be covered by one another.

The robot arm 105 is a device capable of moving and rotating the ultrasound probe 101. For example, the robot arm 105 is configured to grip the ultrasound probe 101 in a tip end part thereof. Further, under control of robot arm controlling circuitry 170 (explained later), the robot arm 105 is configured to control movement of scan cross-sectional planes, by moving the gripped ultrasound probe 101 along the body surface of the patient P and rotating the gripped ultrasound probe 101 on the body surface.

Further, the robot arm 105 includes a pressure sensor and is configured to detect contact pressure (body surface contact pressure) of the ultrasound probe 101 against the body surface. The detected body surface contact pressure is transmitted to the robot arm controlling circuitry 170 (explained later) and is monitored so as to maintain appropriate values for safety.

The above description of the robot arm 105 is merely an example. It is possible to select and adopt, as appropriate, any of publicly-known techniques regarding the robot arm 105 configured to control the movement of the ultrasound probe 101. Further, in the example in FIG. 1, the robot arm 105 and the apparatus main body 100 are integrally formed; however, the robot arm 105 and the apparatus main body 100 may be configured separately.

The apparatus main body 100 is an apparatus configured to generate ultrasound image data on the basis of the reflected-wave signals received by the ultrasound probe 101. As illustrated in FIG. 1, the apparatus main body 100 includes transmission and reception circuitry 110, signal processing circuitry 120, image generating circuitry 130, an image memory 140, storage circuitry 150, processing circuitry 160, and the robot arm controlling circuitry 170. The transmission and reception circuitry 110, the signal processing circuitry 120, the image generating circuitry 130, the image memory 140, the storage circuitry 150, the processing circuitry 160, and the robot arm controlling circuitry 170 are connected so as to be able to communicate with one another.

The transmission and reception circuitry 110 is configured to perform an ultrasound wave scanning (an ultrasound scan) by controlling the ultrasound probe 101. The transmission and reception circuitry 110 includes a pulse generator, a transmission delay unit, a pulser, and the like and is configured to supply the drive signal to the ultrasound probe 101. The pulse generator is configured to repeatedly generate a rate pulse for forming a transmission ultrasound wave at a predetermined rate frequency. Further, the transmission delay unit is configured to apply a delay time period that is required to converge the ultrasound waves generated by the ultrasound probe 101 into the form of a beam and to determine transmission directionality and that corresponds to each of the piezoelectric transducer elements, to each of the rate pulses generated by the pulse generator. Also, the pulser is configured to apply the drive signal (a drive pulse) to the ultrasound probe 101 with timing based on the rate pulses. In other words, by varying the delay time periods applied to the rate pulses, the transmission delay unit is able to arbitrarily adjust the transmission directions of the ultrasound waves transmitted from the surfaces of the piezoelectric transducer elements.

In this situation, the transmission and reception circuitry 110 has a function that is able to instantly change transmission frequencies, transmission drive voltage, and the like, for the purpose of executing a predetermined scan sequence on the basis of an instruction from the processing circuitry 160 (explained later). In particular, the function to change the transmission drive voltage is realized by using linear-amplifier-type transmission circuitry of which the value can be instantly switched or by using a mechanism configured to electrically switch between a plurality of power source units.

Further, the transmission and reception circuitry 110 includes a pre-amplifier, an Analog/Digital (A/D) converter, a reception delay unit, an adder, and the like and is configured to generate reflected-wave data by performing various types of processes on the reflected-wave signals received by the ultrasound probe 101. The pre-amplifier is configured to amplify the reflected-wave signal for each of the channels. The A/D converter is configured to perform an A/D conversion on the amplified reflected-wave signals. The reception delay unit is configured to apply a delay time period required to determine reception directionality. The adder is configured to generate the reflected-wave data by performing an adding process on the reflected-wave signals processed by the reception delay unit. As a result of the adding process performed by the adder, reflected components of the reflected-wave signals that are from the direction corresponding to the reception directionality are emphasized, so that a comprehensive beam used in the ultrasound wave transmission and reception is formed on the basis of the reception directionality and the transmission directionality.

When a two-dimensional region of the patient P is to be scanned, the transmission and reception circuitry 110 causes an ultrasound beam to be transmitted from the ultrasound probe 101 in a two-dimensional direction. After that, the transmission and reception circuitry 110 generates two-dimensional reflected-wave data from reflected-wave signals received by the ultrasound probe 101. In contrast, when a three-dimensional region of the patient P is to be scanned, the transmission and reception circuitry 110 causes an ultrasound beam to be transmitted from the ultrasound probe 101 in a three-dimensional direction. After that, the transmission and reception circuitry 110 generates three-dimensional reflected-wave data from reflected-wave signals received by the ultrasound probe 101.

For example, the signal processing circuitry 120 is configured to generate data (B-mode data) in which the signal intensity at each sampling point is expressed by a degree of brightness, by performing a logarithmic amplification, an envelope detecting process, and/or the like on the reflected-wave data received from the transmission and reception circuitry 110. The B-mode data generated by the signal processing circuitry 120 is output to the image generating circuitry 130. The B-mode data is an example of scan data.

Further, for example, the signal processing circuitry 120 is configured to generate data (Doppler data) obtained by extracting movement information of the moving members based on the Doppler effect at each of the sampling points within a scanned region, from the reflected-wave data received from the transmission and reception circuitry 110. More specifically, the signal processing circuitry 120 is configured to generate data (Doppler data) obtained by extracting moving member information such as an average velocity value, dispersion, power, and the like with respect to multiple points, by performing a frequency analysis to obtain velocity information from the reflected-wave data and extracting a blood flow, a tissue, and a contrast agent echo component subject to the Doppler effect. In this situation, the moving members are, for example, blood flows, tissues such as the cardiac wall, and a contrast agent. The movement information (blood flow information) obtained by the signal processing circuitry 120 is sent to the image generating circuitry 130 and is displayed in color on the display device 103, as an average velocity image, a dispersion image, a power image, or an image combining any of these images. The Doppler data is an example of scan data.

The image generating circuitry 130 is configured to generate ultrasound image data from the data generated by the signal processing circuitry 120. The image generating circuitry 130 is configured to generate B-mode image data in which the intensities of the reflected waves are expressed as brightness levels, from the B-mode data generated by the signal processing circuitry 120. Further, the image generating circuitry 130 is configured to generate Doppler image data indicating the moving member information, from the Doppler data generated by the signal processing circuitry 120. The Doppler image data is velocity image data, dispersion image data, power image data, or image data combining together any of these types of image data.

In this situation, generally speaking, the image generating circuitry 130 converts (by performing a scan convert process) a scanning line signal sequence from an ultrasound scan into a scanning line signal sequence in a video format used by, for example, television and generates display-purpose ultrasound image data. More specifically, the image generating circuitry 130 generates the display-purpose ultrasound image data by performing a coordinate transformation process compliant with the ultrasound scanning mode used by the ultrasound probe 101. Further, as various types of image processing processes besides the scan convert process, the image generating circuitry 130 performs, for example, an image processing process (a smoothing process) to re-generate an average brightness value image, an image processing process (an edge enhancement process) that uses a differential filter inside an image, or the like, by using a plurality of image frames resulting from the scan convert process. Also, the image generating circuitry 130 combines additional information (text information of various types of parameters, scale graduations, body marks, and the like) with the ultrasound image data.

In other words, the B-mode data and the Doppler data are each ultrasound image data before the scan convert process. The data generated by the image generating circuitry 130 is the display-purpose ultrasound image data after the scan convert process. When the signal processing circuitry 120 has generated three-dimensional scan data (three-dimensional B-mode data and three-dimensional Doppler data), the image generating circuitry 130 generates volume data by performing a coordinate transformation process compliant with the ultrasound scanning mode used by the ultrasound probe 101. Further, the image generating circuitry 130 generates display-purpose two-dimensional image data, by performing various types of rendering processes on the volume data.

The image memory 140 is a memory configured to store therein the display-purpose image data (a display-purpose image) generated by the image generating circuitry 130. Further, the image memory 140 is also capable of storing therein any of the data generated by the signal processing circuitry 120. After a diagnosis process, for example, the operator is able to invoke any of the B-mode data and the Doppler data stored in the image memory 140. The invoked data can serve as the display-purpose ultrasound image data after being routed through the image generating circuitry 130.

The storage circuitry 150 is configured to store therein a control program for performing ultrasound wave transmissions and receptions, image processing processes, and display processes, as well as diagnosis information (e.g., patients' IDs and observations of medical doctors) and various types of data such as diagnosis protocols, various types of body marks, and the like. Further, as necessary, the storage circuitry 150 may be used for saving therein any of the image data stored in the image memory 140. Further, it is also possible to transfer any of the data stored in the storage circuitry 150 to an external device via an interface (not illustrated).

The processing circuitry 160 is configured to control the entirety of processes performed by the ultrasound diagnosis apparatus 1. More specifically, on the basis of the various types of setting requests input by the operator via the input interface 102 and various types of control programs and various types of data read from the storage circuitry 150, the processing circuitry 160 is configured to control processes performed by the transmission and reception circuitry 110, the signal processing circuitry 120, the image generating circuitry 130, and the robot arm controlling circuitry 170. Further, the processing circuitry 160 is configured to exercise control so that the display device 103 displays the display-purpose ultrasound image data stored in the image memory 140.

Further, as illustrated in FIG. 1, the processing circuitry 160 is configured to execute the scan controlling function 161 and an evaluating function 162. The scan controlling function 161 is an example of a scan controlling unit. The evaluating function 162 is an example of an evaluating unit.

In this situation, for example, the processing functions executed by the constituent elements of the processing circuitry 160 illustrated in FIG. 1, namely, the scan controlling function 161 and the evaluating function 162 are recorded in a storage device (e.g., the storage circuitry 150) of the ultrasound diagnosis apparatus 1 in the form of computer-executable programs. The processing circuitry 160 is a processor configured to realize the functions corresponding to the programs by reading and executing the programs from the storage device. In other words, the processing circuitry 160 that has read the programs has the functions illustrated within the processing circuitry 160 in FIG. 1. The processing functions executed by the scan controlling function 161 and the evaluating function 162 will be explained later.

The robot arm controlling circuitry 170 is configured to control motion of the robot arm 105. As for the robot arm controlling circuitry 170, for example, the robot arm controlling circuitry 170 is configured to move the ultrasound probe 101 to a desired position (an initial position) by driving the robot arm 105 in accordance with control exercised by the processing circuitry 160 (the scan controlling function 161).

Further, on the basis of a motion condition of the robot arm 105, the robot arm controlling circuitry 170 is configured to control the motion of the robot arm 105. The motion condition may be information indicating a position (the initial position) in which a scan is to be started or information defining a procedure to move/rotate the ultrasound probe 101 starting at the initial position. As the motion condition, information being pre-set for each site or cross-sectional plane serving as a scan target is stored in the storage circuitry 150 in advance. Further, the pre-setting of the motion condition may be changed as appropriate by the operator (a user).

Further, to move the ultrasound probe 101 safely, the robot arm controlling circuitry 170 has a function of avoiding obstacles and a function of maintaining the body surface contact pressure. For example, the robot arm controlling circuitry 170 is configured to detect the obstacles on traveling paths and unevenness on the body surface from the camera image data taken by the camera 104. Further, the robot arm controlling circuitry 170 is configured to change the traveling paths so as to avoid the detected obstacles and to move along the detected unevenness on the body surface. Also, the robot arm controlling circuitry 170 is configured to monitor the body surface contact pressure obtained from the pressure sensor included in the robot arm 105. Further, the robot arm controlling circuitry 170 is configured to move the ultrasound probe 101 on a traveling path while adjusting the position thereof in the pressing direction (the direction perpendicular to the body surface) so that the body surface contact pressure is maintained at values within a predetermined range. With these arrangements, the robot arm controlling circuitry 170 is able to move the ultrasound probe 101 along the traveling path, while preventing excessive pressure from being applied to the body surface and preventing the ultrasound probe 101 from coming out of contact with the body surface. As for the motion control exercised by the robot arm controlling circuitry 170 on the robot arm 105, it is possible to arbitrary adopt any of publicly-known motion control techniques besides the one described above.

The term “processor (circuitry)” used in the above description denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processor is configured to realize the functions by reading and executing the programs saved in the storage circuitry 150. Alternatively, instead of having the programs saved in the storage circuitry 150, it is also acceptable to directly incorporate the programs in the circuitry of the processor. In that situation, the processor realizes the functions by reading and executing the programs incorporated in the circuitry thereof. Further, the processors of the present embodiment do not each necessarily have to be structured as a single piece of circuitry. It is also acceptable to structure one processor by combining together a plurality pieces of independent circuitry so as to realize the functions thereof. Further, it is also acceptable to integrate two or more of the constituent elements illustrated in any of the drawings into one processor so as to realize the functions thereof.

A basic configuration of the ultrasound diagnosis apparatus 1 according to the present embodiment has thus been explained. The ultrasound diagnosis apparatus 1 according to the present embodiment structured as described above is configured to perform the processes described below, so as to obtain an image having desired quality, by performing a scan that uses the robot arm 105.

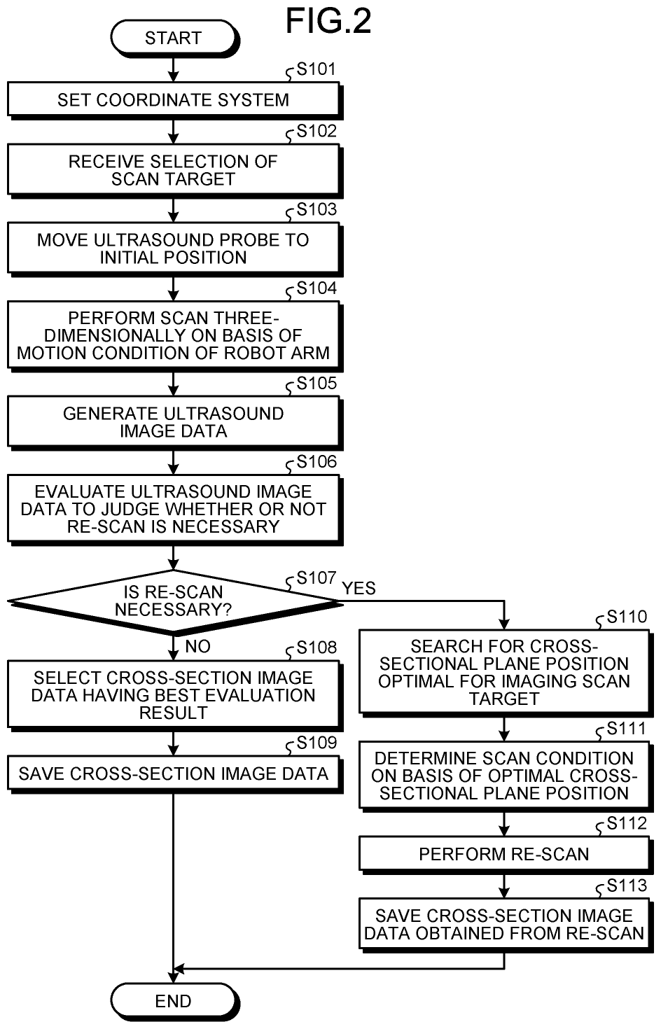

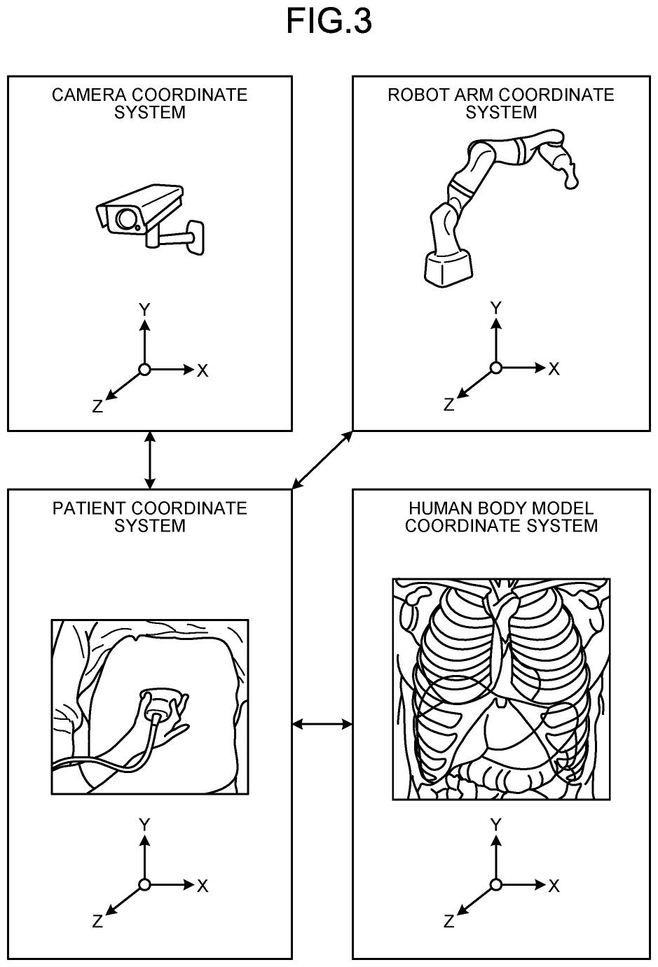

A processing procedure performed by the ultrasound diagnosis apparatus 1 according to an embodiment will be explained, with reference to FIG. 2. FIG. 2 is a flowchart illustrating the processing procedure performed by the ultrasound diagnosis apparatus 1 according to the embodiment. With FIG. 2, the procedure will be described with reference to FIGS. 3 to 8.

The processing procedure in FIG. 2 is started when the operator inputs an instruction to start an imaging process (a scan) that uses the robot arm 105, for example. Until the instruction to start the imaging process that uses the robot arm 105 is input, the processes in FIG. 2 will not be started and are in a standby state. Further, the processing procedure in FIG. 2 does not necessarily have to be performed in the order illustrated in FIG. 2. It is possible to arbitrary change the order, as long as no conflict occurs in the processes.

As illustrated in FIG. 2, when the operator inputs an instruction to start an imaging process that uses the robot arm 105, the ultrasound diagnosis apparatus 1 starts the processes at step S101 and thereafter. Unless an instruction to start an imaging process is input, the processes at step S101 and thereafter will not be started, and the processes in FIG. 2 are in a standby state.

The scan controlling function 161 sets a coordinate system (step S101). For example, the scan controlling function 161 sets a patient coordinate system, a camera coordinate system, a robot arm coordinate system, and a human body model coordinate system. After that, as illustrated in FIG. 3, the scan controlling function 161 brings the camera coordinate system, the robot arm coordinate system, and the human body model coordinate system into association with the patient coordinate system. FIG. 3 is a drawing for explaining the coordinate systems according to the present embodiment.

In this situation, the patient coordinate system is a coordinate system of real space in which the patient is present. For example, the patient coordinate system is a coordinate system of which the origin is at the solar plexus (the epigastrium) of the patient P lying down, of which the Z-axis direction corresponds to the body axis direction (the longitudinal direction of the examination table), of which the Y-axis direction corresponds to the vertical direction (the gravity direction), and of which the X-axis direction corresponds to the direction orthogonal to the Y-axis direction and to the Z-axis directions. For example, the operator designates the position of the solar plexus and the axial directions. On the basis of the position of the solar plexus and the axial directions designated by the operator, the scan controlling function 161 is configured to set the patient coordinate system.

The camera coordinate system is a coordinate system of the camera image data taken by the camera 104. The coordinate system of the camera image data is two-dimensional coordinate system; however, it is possible to estimate depth information by designating the distances from the lens position of the camera 104 to each of representative structures such as the floor surface, wall surfaces, and the examination table. For example, the operator designates coordinates in the patient coordinate system with respect to arbitrary points (preferably three or more points) rendered in the camera image data. By using the coordinates of the points designated by the operator, the scan controlling function 161 brings the camera coordinate system into association with the patient coordinate system.

The robot arm coordinate system is a coordinate system expressing the movable range of the robot arm 105. For example, the robot arm coordinate system uses the installation position of the robot arm 105 as the origin and is defined by the lengths and the rotation angles of the arms.

For example, the operator designates coordinates in the patient coordinate system with respect to arbitrary points (preferably three or more points) included in the robot arm 105. By using the coordinates of the points designated by the operator, the scan controlling function 161 brings the robot arm coordinate system into association with the patient coordinate system.

The human body model coordinate system is a coordinate system expressing three-dimensional positions (coordinates) of different parts included in a human body model. In the present example, the human body model is information indicating the positions of representative organs, bones, joints, skin (the body surface), and the like of a standard human body. For example, the operator designates coordinates in the patient coordinate system with respect to arbitrary points (preferably three or more points) included in the human body model. By using the coordinates of the points designated by the operator, the scan controlling function 161 brings the human body model coordinate system into association with the patient coordinate system.

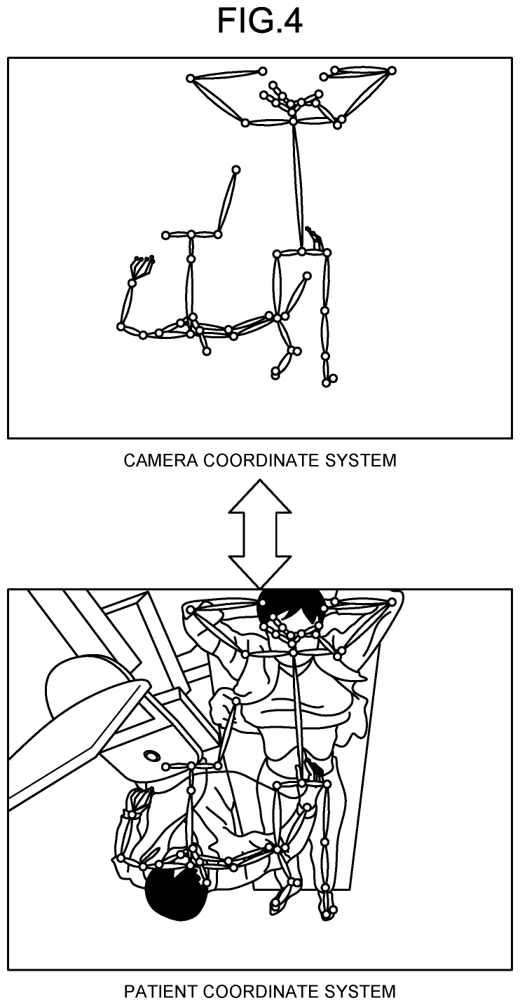

As explained above, the scan controlling function 161 is configured to set the coordinate systems and to bring the coordinate systems into association with one another. As a result, for example, as illustrated in FIG. 4, it is also possible to bring the skeleton information (the top section of FIG. 4) obtained from the camera image data into association with the patient coordinate system (the bottom section of FIG. 4) and the other coordinate systems. FIG. 4 is a drawing for explaining the association between the skeleton information and the patient coordinate system according to the embodiment.

The scan controlling function 161 receives selection of a scan target (step S102). For example, the operator performs an operation to select a desired site. The scan controlling function 161 receives the site selected by the operator as the scan target. As a specific example, the operator performs an operation to select the “gall bladder”. The scan controlling function 161 receives the “gall bladder” selected by the operator, as the scan target.

What can be selected as the scan target does not necessarily have to be a site and may be a cross-sectional plane, for example. For instance, when the operator selects an apical four-chamber view (an A4C view) as a desired cross-sectional plane, the scan controlling function 161 receives the “A4C view” as the scan target. Further, the “gall bladder” and the “A4C view” described above are merely examples of the scan target, and it is possible to set an arbitrary site (e.g., an organ or a structure) or an arbitrary cross-sectional plane as the scan target.

The scan controlling function 161 moves the ultrasound probe 101 to the initial position (step S103). In this situation, the initial position is position information indicating an abutment position on the body surface against which the ultrasound probe 101 is caused to abut, at the starting time of the scan, as well as the orientation of the ultrasound probe 101 in the abutment position.

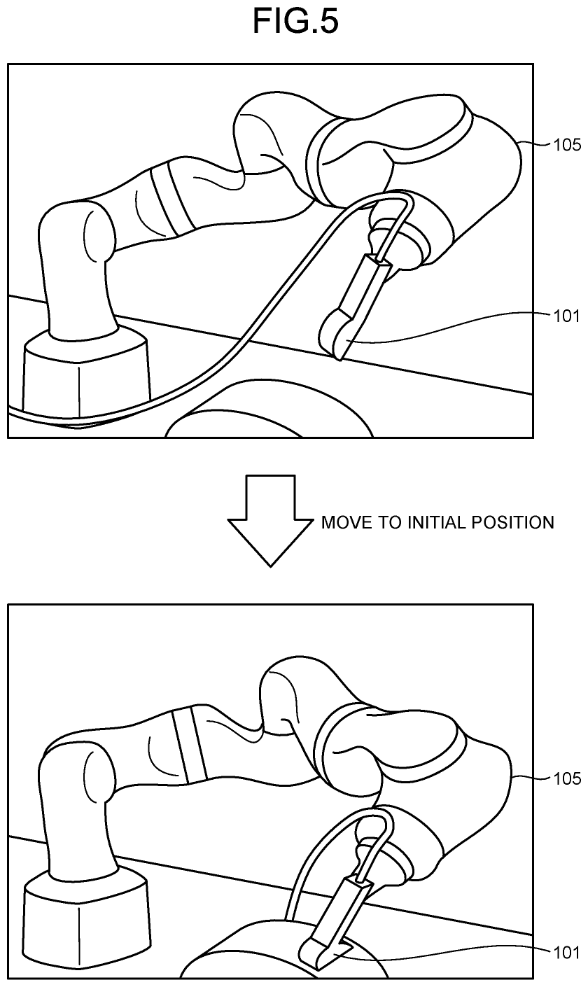

For example, the scan controlling function 161 releases the joints of the robot arm 105 from a fixation state. After that, as illustrated in FIG. 5, the operator moves the ultrasound probe 101 gripped at the tip end part of the robot arm 105 to the initial position with a manual operation and inputs an instruction to fix the position of the ultrasound probe 101. The scan controlling function 161 recognizes the position fixed according to the instruction from the operator, as the initial position. FIG. 5 is a drawing for explaining the setting of the initial position according to the embodiment.

Alternatively, the ultrasound probe 101 does not necessarily have to be moved to the initial position by the manual operation and may automatically be moved by the robot arm 105. In that situation, for example, the storage circuitry 150 has stored therein information indicating an initial position for each of various scan targets. The scan controlling function 161 reads information indicating the initial position corresponding to the scan target selected by the operator, from the storage circuitry 150. After that, on the basis of the association between the patient coordinate system and the robot arm coordinate system, the scan controlling function 161 transforms the read information indicating the initial position into position information in the robot arm coordinate system. On the basis of the information indicating the initial position and having been transformed into the robot arm coordinate system by the scan controlling function 161, the robot arm controlling circuitry 170 puts the robot arm 105 in motion. In this manner, the robot arm controlling circuitry 170 moves the ultrasound probe 101 gripped by the robot arm 105 from a present position to the initial position.

On the basis of the motion condition of the robot arm 105, the scan controlling function 161 performs a scan three-dimensionally (step S104). In this situation, the motion condition is information set in advance for each of the various scan targets. For example, the motion condition is stored in a motion condition table within the storage circuitry 150.

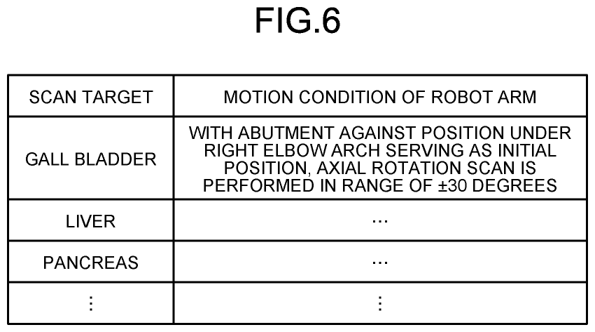

The motion condition table according to the embodiment will be explained with reference to FIG. 6. FIG. 6 is a drawing for explaining the motion condition table according to the embodiment.

As illustrated in FIG. 6, the motion condition table has stored therein information in which “scan targets” are kept in correspondence with “motion conditions of the robot arm”. The scan targets are each represented by information indicating a site or a cross-sectional plane serving as a scan target. The motion conditions of the robot arm are each represented by either information indicating a position in which the scan is to be started (the initial position) or information defining a procedure to move/rotate the ultrasound probe 101 (a trajectory of the movement) starting at the initial position.

For example, the motion condition table has stored therein the information in which the scan target “gall bladder” is kept in correspondence with the motion condition of the robot arm indicating that “With abutment against a position under the right rib bow serving as an initial position, an axial rotation scan is performed in the range of ±30 degrees”. The information indicates that, when the scan target is the “gall bladder”, an axial rotation scan is to be performed in the range of ±30 degrees while the ultrasound probe 101 is caused to abut while using a position underneath the right rib bow as the initial position. In the present example, the axial rotation scan denotes a scan method by which the ultrasound probe 101 is moved so as to flap the scan cross-sectional plane by being tilted while the abutment position of the ultrasound probe 101 is used as a fulcrum. Also, the motion condition table has similarly stored therein the other scan targets so as to be kept in correspondence with motion conditions of the robot arm.

The explanation with reference to FIG. 6 is merely an example, and possible embodiments are not limited to this example. For instance, it is possible to set, as the scan target, an arbitrary site or an arbitrary cross-sectional plane other than the “gall bladder”. Further, it is possible to set the motion condition of the robot arm 105 corresponding to each of the scan targets on the basis of, for example, a manual or a guideline created by any of various types of medical institutions and academic societies.

Further, the motion condition of the robot arm is capable of defining not only the axial rotation scan, but also a parallel or a complex scan in which an axial rotation scan is combined with a parallel scan, and the like. In this situation, the parallel scan denotes a scan method by which the ultrasound probe 101 is moved while the orientation of the ultrasound probe 101 is fixed. It is also possible to combine an axial rotation scan with a parallel scan.

From the motion condition table stored in the storage circuitry 150, the scan controlling function 161 reads the “motion condition of the robot arm” corresponding to the “scan target” selected by the operator. Further, the scan controlling function 161 performs the scan on the basis of the read motion condition.

For example, while causing the ultrasound probe 101 to perform ultrasound wave transmission and reception, the scan controlling function 161 causes the robot arm controlling circuitry 170 to carry out the movement defined by the motion condition. In other words, the scan controlling function 161 is configured to have a three-dimensional region (space) scanned, by causing the ultrasound probe 101 to perform the scan while being moved on the scan cross-sectional plane on the basis of the motion condition. The three-dimensional space is expressed as reflected-wave data from a plurality of scan cross-sectional planes in mutually-different positions.

Further, during the motion of the ultrasound probe 101, the scan controlling function 161 is configured to correct the position of the ultrasound probe 101, on the basis of at least one selected from among: the skeleton information of the patient P, body surface information of the patient P, scan angle information of the ultrasound probe 101, contact pressure information of the ultrasound probe 101, cardiac phase information of the patient P, and respiratory phase information of the patient P. In this situation, the skeleton information is information indicating the positions of a plurality of representative joints of the patient P and the positions of representative bones connecting the joints and is obtained from color image data. The body surface information is information indicating the shape of the body surface of the patient P and is estimated on the basis of the skeleton information and information about the body surface of the human body model. The scan angle information is information indicating an abutment angle of the ultrasound probe 101 against the body surface and is estimated on the basis of the body surface information and the position information of the ultrasound probe 101. The contact pressure information is information indicating the body surface contact pressure and is obtained by the pressure sensor installed on the robot arm 105. The cardiac phase information is obtained from an electrocardiogram (ECG). The respiratory phase information is obtained by a respiration monitoring device or from camera image data, while a voice message prompts the patient to hold his/her breath. For example, to avoid obstacles and to maintain the body surface contact pressure, the scan controlling function 161 corrects the position of the ultrasound probe 101, by controlling the robot arm controlling circuitry 170 on the basis of at least one selected from among: the skeleton information, the body surface information, the scan angle information, the contact pressure information, the cardiac phase information, and the respiratory phase information.

The image generating circuitry 130 generates ultrasound image data (step S105). For example, on the basis of the reflected-wave data from the plurality of scan cross-sectional planes structuring the three-dimensional region, the image generating circuitry 130 generates “a plurality of pieces of cross-section image data” respectively corresponding to the plurality of scan cross-sectional planes. Further, the image generating circuitry 130 generates “volume data” by incorporating the reflected-wave data from the plurality of scan cross-sectional planes into a pre-defined three-dimensional data space while performing an interpolation process (a coordinate transformation process). In the present embodiment, the plurality of pieces of cross-section image data representing the three-dimensional region and the volume data are inclusively referred to as “ultrasound image data”.

In this situation, the image generating circuitry 130 does not necessarily have to generate both the plurality of pieces of cross-section image data and the volume data. For example, the image generating circuitry 130 may generate, as necessary, one or both of the plurality of pieces of cross-section image data and the volume data.

The evaluating function 162 evaluates the ultrasound image data so as to judge whether or not it is necessary to perform a re-scan (step S106). For example, the evaluating function 162 judges whether or not the re-scan is necessary, on the basis of one or both of: the extent to which the scan target is rendered in the ultrasound image data; and image quality of the ultrasound image data. For example, as the extent of the rendering, the evaluating function 162 may use one or both of: a degree of similarity between the scan target in the ultrasound image data and representative image data of the scan target; and the size of the scan target. Further, as the image quality, the evaluating function 162 may use the size of a low echo-signal region in the ultrasound image data.

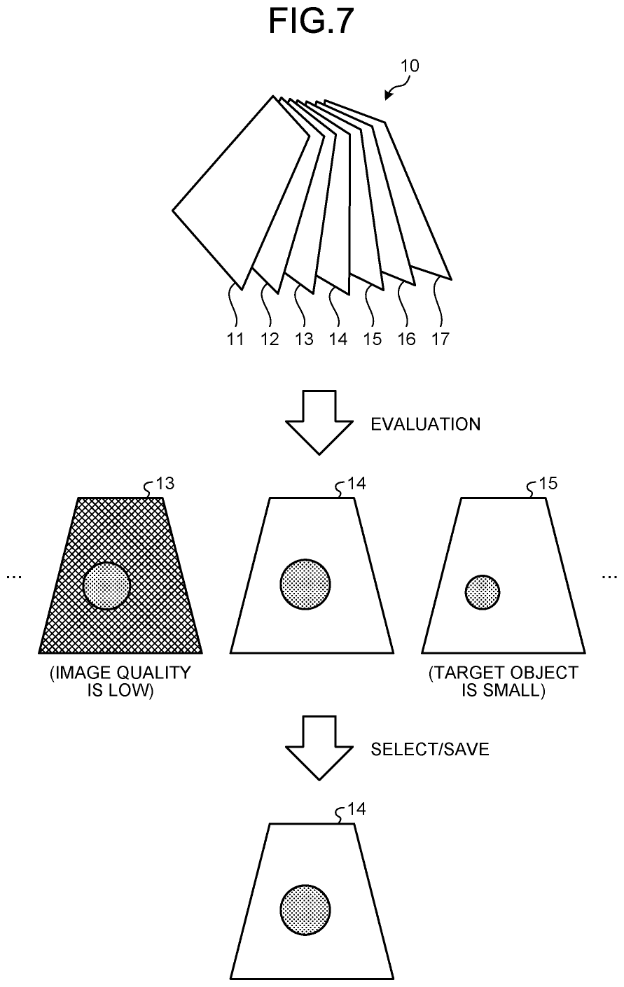

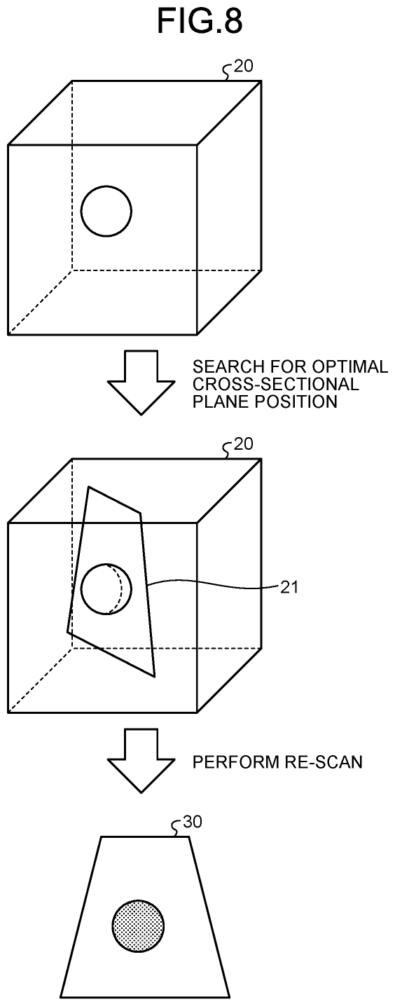

Processes performed by the evaluating function 162 according to the embodiment will be explained, with reference to FIGS. 7 and 8. FIGS. 7 and 8 are drawings for explaining the processes performed by the evaluating function 162 according to the embodiment. In FIGS. 7 and 8, the circles in the images each represent the structure designated as the scan target.

With reference to FIG. 7, an example will be explained in which a cross-section image data group 10 has been generated by performing a scan using the robot arm 105. As illustrated in the top section of FIG. 7, the cross-section image data group 10 is an image data group corresponding to a three-dimensional region and includes seven pieces of cross-section image data 11, 12, 13, 14, 15, 16, and 17. The evaluating function 162 is configured to calculate levels of similarity between the structure (e.g., the gall bladder) rendered in the pieces of cross-section image data 11 to 17 and a representative (standard) piece of image data of the structure. Further, the evaluating function 162 is configured to calculate the largest diameter, as the size of the structure rendered in the pieces of cross-section image data 11 to 17. Also, with respect to the pieces of cross-section image data 11 to 17, the evaluating function 162 is configured to detect the low echo-signal region such as an acoustic shadow region or a low Signal-to-Noise (S/N) ratio region in a deep part and to calculate the area thereof as the size of the detected low echo-signal region. After that, the evaluating function 162 is configured to calculate an evaluation score on the basis of the levels of similarity, the largest diameter, and the area that were calculated. It is possible to calculate the evaluation score by using an arbitrary mathematical function. Further, the evaluating function 162 is configured to judge whether or not a re-scan is necessary, on the basis of whether or not image data having an evaluation score equal to or larger than a predetermined value (a threshold value) is present among the seven pieces of cross-section image data 11 to 17. In this manner, the evaluating function 162 evaluates the ultrasound image data obtained from the scan so as to judge whether or not it is necessary to perform the re-scan using the robot arm 105.

For example, as illustrated in the middle section of FIG. 7, when the pieces of cross-section image data 13, 14, and 15 each have an evaluation score equal to or larger than the predetermined value, the evaluating function 162 determines that the re-scan is unnecessary (step S107: No). Further, the evaluating function 162 selects ultrasound image data having the best evaluation result (step S108). For example, the piece of cross-section image data 13 has lower image quality than the pieces of cross-section image data 14 and 15. Also, in the piece of cross-section image data 15, the structure is rendered smaller than in the pieces of cross-section image data 13 and 14. In that situation, the evaluation score of the piece of cross-section image data 14 is the best score. Accordingly, the evaluating function 162 selects the piece of cross-section image data 14, as illustrated in the bottom section of FIG. 7.

After that, the evaluating function 162 saves the selected ultrasound image data (step S109). For example, the evaluating function 162 saves the piece of cross-section image data 14 selected in the bottom section of FIG. 7 into a predetermined storage unit (e.g., the storage circuitry 150). In other words, when the re-scan is unnecessary, the evaluating function 162 saves the ultrasound image data having the best evaluation result into the predetermined storage unit.

The description with reference to FIG. 7 is merely an example, and possible embodiments are not limited to the example in FIG. 7. For instance, although FIG. 7 illustrates the example in which the scan target is a “site”, when the scan target is a “cross-sectional plane”, the evaluating function 162 is able to calculate, as the degree to which the scan target is rendered, levels of similarity between the pieces of cross-section image data obtained from the scan and a representative image of the cross-sectional plane.

Further, the function of calculating the degree of the rendering and the image quality is not limited to the processes described above. It is also possible to provide the degree of the rendering and the image quality by using a trained model that has been trained in advance by machine learning. Further, the function of calculating the degree of the rendering and the image quality is not limited to the processes described above, and it is possible to arbitrarily adopt any of publicly-known techniques.

On the contrary, when no image data having an evaluation score equal to or larger than the predetermined value (the threshold value) is present among the seven pieces of cross-section image data 11 to 17, the evaluating function 162 determines that the re-scan is necessary (step S107: Yes). After that, the evaluating function 162 searches for a cross-sectional plane position optimal for imaging the scan target (step S110). For example, as illustrated in the top section of FIG. 8, the evaluating function 162 searches for the optimal cross-sectional plane position, by using volume data 20 corresponding to the three-dimensional region. More specifically, as the optimal cross-sectional plane position, the evaluating function 162 searches for a scan cross-sectional plane 21 that passes through the position on the body surface closest to the structure and that renders the structure in the largest size (the middle section of FIG. 8). As explained herein, when the re-scan is necessary, the evaluating function 162 searches for the optimal cross-sectional plane position in the ultrasound image data.

Further, when the scan cross-sectional plane 21 found in the search is shadowed by gas (air) or a bone, the evaluating function 162 is configured to correct (adjust) the scan cross-sectional plane 21. For example, by referring to the skeleton information of the patient P and human body model information, the evaluating function 162 judges whether or not the scan cross-sectional plane 21 is shadowed by gas or a bone. In this situation, for example, when the scan cross-sectional plane 21 is shadowed by a rib, the top side (the abutment position of the ultrasound probe 101) of the scan cross-sectional plane 21 is moved to another position where there is no shadow of the ribs. After that, the evaluating function 162 corrects the scan cross-sectional plane 21 so as to pass through the abutment position of the ultrasound probe 101 that has been moved and to render the structure in large size. The evaluating function 162 updates by using the corrected scan cross-sectional plane as an optimal scan cross-sectional plane.

Subsequently, on the basis of the optimal cross-sectional plane position, the scan controlling function 161 determines a scan condition (step S111). For example, the scan controlling function 161 calculates position information indicating the position and the orientation of the ultrasound probe 101 for scanning the scan cross-sectional plane 21 illustrated in the middle section of FIG. 8.

Additionally, the scan controlling function 161 is capable of determining not only the position information of the ultrasound probe 101 but also cardiac phase information or respiratory phase information as a scan condition. As for the cardiac phase information and the respiratory phase information, an appropriate temporal phase is defined in advance for each of the scan targets and is stored in the storage circuitry 150, for example.

Further, the scan controlling function 161 performs the re-scan (step S112). For example, the scan controlling function 161 is configured to move the ultrasound probe 101 to the position in which the scan cross-sectional plane 21 is scanned, by controlling the robot arm controlling circuitry 170. Further, the scan controlling function 161 is configured to move the ultrasound probe 101 to the position where the scanning of the scan cross-sectional plane 21 is possible, by controlling the robot arm controlling circuitry 170 on the basis of the position information of the ultrasound probe 101 calculated as the scan condition. In this situation, to avoid obstacles and maintain the body surface contact pressure during the motion of the robot arm 105, the scan controlling function 161 is configured to correct the position of the ultrasound probe 101, by controlling the robot arm controlling circuitry 170 on the basis of at least one selected from among: the skeleton information, the body surface information, the scan angle information, the contact pressure information, the cardiac phase information, and the respiratory phase information.

Further, the scan controlling function 161 performs the re-scan by causing the ultrasound probe 101 to perform ultrasound wave transmission and reception in the position to which the ultrasound probe 101 has been moved. As explained herein, the scan controlling function 161 performs the re-scan on the basis of the optimal scan cross-sectional plane found in the search.

When the cardiac phase information or the respiratory phase information is defined as the scan condition, the scan controlling function 161 is configured to control the ultrasound probe 101 so as to perform ultrasound wave transmission and reception in a defined temporal phase. Accordingly, the scan controlling function 161 performs the re-scan with timing corresponding to the cardiac phase information or the respiratory phase information being defined. As a result, as illustrated in the bottom section of FIG. 8, cross-section image data 30 corresponding to the scan cross-sectional plane 21 is generated.

After that, the evaluating function 162 saves the ultrasound image data obtained from the re-scan (step S113). For example, with respect to the cross-section image data 30 obtained from the re-scan, the evaluating function 162 calculates an evaluation score explained above and confirms that the evaluation score is equal to or larger than the predetermined value, before saving the cross-section image data 30 into the predetermined storage unit. In this situation, when the evaluation score is smaller than the predetermined value, the evaluating function 162 is able to have a re-scan performed again.

The description with reference to FIG. 8 is merely an example, and possible embodiments are not limited to the description illustrated in FIG. 8. For example, the process of searching for the optimal scan cross-sectional plane is not limited to the description above. It is possible to arbitrarily adopt any of publicly-known searching techniques. Further, with respect to the cross-section image data 30 obtained from the re-scan, the evaluation score does not necessarily have to be calculated. For example, the evaluating function 162 may save the cross-section image data 30 into the predetermined storage unit, without performing any additional process.

When the ultrasound image data has been saved in the predetermined storage unit, the processing circuitry 160 ends the process in FIG. 2. The processing procedure illustrated in FIG. 2 does not necessarily have to be performed in the order indicated in FIG. 2. It is possible to arbitrarily make changes as long as no conflict occurs in the processes.

As explained above, the ultrasound diagnosis apparatus 1 according to the present embodiment includes the robot arm 105, the scan controlling function 161, and the evaluating function 162. The robot arm 105 is capable of moving and rotating the ultrasound probe 101. The scan controlling function 161 is configured to three-dimensionally perform the scan by using the robot arm 105. The evaluating function 162 is configured to evaluate the ultrasound image data obtained from the scan, so as to judge whether or not it is necessary to perform a re-scan using the robot arm 105. As a result, the ultrasound diagnosis apparatus 1 is able to obtain the image having desired quality, by performing the scan using the robot arm 105.

For example, existing ultrasound diagnosis apparatuses having a robot arm are configured to perform a scan while moving the robot arm according to a path (a trajectory) defined for each scan target. However, because the shape of the body surface and positional relationships between organs vary among patients (examined subjects), the standardized method may not work in obtaining an image of desired quality, in some situations.

In contrast, the ultrasound diagnosis apparatus 1 according to the present embodiment is configured to evaluate the quality of the obtained ultrasound image data and is configured, when the desired quality is not satisfied, to perform the re-scan. Further, the ultrasound diagnosis apparatus 1 is configured to search for the optimal scan cross-sectional plane in the ultrasound image data obtained from the first-time scan and to perform the re-scan on the scan cross-sectional plane found in the search. In other words, the ultrasound diagnosis apparatus 1 is configured to search for the more appropriate scan cross-sectional plane, in the same manner as human beings (operators) gradually narrow down a scan range while looking at scan images displayed in a real-time manner. As a result, the ultrasound diagnosis apparatus 1 makes it possible to obtain images having higher quality.

In the embodiment described above, the example was explained in which the plurality of pieces of cross-section image data and the volume data are used as the processing targets; however, possible embodiment are not limited to this example. For instance, the processes performed by the evaluating function 162 described above may be performed on the data prior to the scan convert process (e.g., B-mode data), in an example. In other words, the evaluating function 162 may perform the processes on the “ultrasound data” including the ultrasound image data and the data prior to the scan convert process.

### OTHER EMBODIMENTS

Besides the embodiments described above, it is possible to carry out the present disclosure in various other modes. Adjusting the contact pressure on the basis of pain

In the embodiment described above, the example was explained in which the robot arm 105 is configured to detect the contact pressure (the body surface contact pressure) and to be monitored so as to maintain appropriate contact pressure for safety. However, even when the ultrasound probe 101 is caused to abut at a certain contact pressure level, the pain occurring at the abutment site may vary among individual patients or among different abutment sites. To cope with this situation, the robot arm controlling circuitry 170 may be configured to adjust the contact pressure with which the robot arm 105 keeps the ultrasound probe 101 in contact with the patient P, on the basis of pain occurring in the patient P. In that situation, the robot arm controlling circuitry 170 is configured to function as an “adjusting unit”.

For example, the robot arm controlling circuitry 170 may be configured to estimate the pain occurring in the abutment site of the patient, on the basis of camera image data rendering facial expressions of the patient P. For example, the camera 104 is configured to image the facial expressions of the patient P in a real-time manner, and to generate camera image data rendering the facial expressions of the patient P. Further, the robot arm controlling circuitry 170 is configured to estimate whether or not pain has occurred, by implementing an image recognition technique or the like on the camera image data taken by the camera 104. For example, the robot arm controlling circuitry 170 estimates whether or not pain has occurred, by recognizing changes in the facial expressions caused by the pain (e.g., frowning), while using the image recognition technique. After that, when it is determined that pain has occurred, the robot arm controlling circuitry 170 puts the robot arm 105 in motion with lower contact pressure.

The method for estimating the pain is not limited to detecting the changes in the facial expressions rendered in the camera image data. It is possible to estimate the pain by detecting biological reactions caused by the pain while using any arbitrary method. Examples of the biological reactions caused by the pain include: voice expressing pain, changes in respiration, changes in perspiration, changes in an electrocardiogram, changes in blood pressure, changes in an electromyogram, changes in brain waves, and changes in the pupil diameters.

Further, for example, the constituent elements of the apparatuses and devices in the drawings are based on functional concepts. Thus, it is not necessarily required to physically configure the constituent elements as indicated in the drawings. In other words, specific modes of distribution and integration of the apparatuses and devices are not limited to those illustrated in the drawings. It is acceptable to functionally or physically distribute or integrate all or a part of the apparatuses and devices in any arbitrary units, depending on various loads and the status of use. Further, all or an arbitrary part of the processing functions performed by the apparatuses and devices may be realized by a CPU and a program analyzed and executed by the CPU or may be realized as hardware using wired logic.

With regard to the processes explained in the embodiments described above, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

It is possible to realize the imaging methods explained in the above embodiments, by causing a computer such as a personal computer or a workstation to execute an imaging program prepared in advance. The imaging program may be distributed via a network such as the Internet. Further, the imaging methods may be executed, as being recorded on a computer-readable non-transitory recording medium such as a hard disk, a flexible disk (FD), a Compact Disk Read-Only Memory (CD-ROM), a Magneto Optical (MO) disk, a Digital Versatile Disk (DVD), or the like and being read by a computer from the recording medium.

According to at least one aspect of the embodiments described above, it is possible to obtain the image having the desired quality by performing the scan using the robot arm.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a robot arm capable of moving and rotating an ultrasound probe; and
processing circuitry configured to three-dimensionally perform a scan by using the robot arm and to evaluate ultrasound data obtained from the scan so as to judge whether or not it is necessary to perform a re-scan using the robot arm.

2. The ultrasound diagnosis apparatus according to claim 1, wherein
when it is unnecessary to perform the re-scan, the processing circuitry saves ultrasound data having a best evaluation result into a predetermined storage unit,
when it is necessary to perform the re-scan, the processing circuitry searches for an optimal scan cross-sectional plane in the ultrasound data, and
the processing circuitry performs the re-scan on the basis of the optimal scan cross-sectional plane found in the search.

3. The ultrasound diagnosis apparatus according to claim 1, wherein the processing circuitry judges whether or not it is necessary to perform the re-scan on the basis of one or both of: a degree to which a scan target is rendered in the ultrasound data; and image quality of the ultrasound data.

4. The ultrasound diagnosis apparatus according to claim 3, wherein
as the degree of the rendering, the processing circuitry uses one or both of: a level of similarity between the scan target in the ultrasound data and representative image data of the scan target; and a size of the scan target, and
as the image quality, the processing circuitry uses a size of a low echo-signal region in the ultrasound data.

5. The ultrasound diagnosis apparatus according to claim 1, wherein the processing circuitry performs the scan on the basis of a motion condition set in advance for each scan target.

6. The ultrasound diagnosis apparatus according to claim 1, wherein, on a basis of pain occurring in an examined subject, the processing circuitry further adjusts contact pressure with which the robot arm keeps the ultrasound probe in contact with the examined subject.

7. The ultrasound diagnosis apparatus according to claim 6, wherein, the processing circuitry estimates the pain on a basis of camera image data rendering a facial expression of the examined subject.

8. The ultrasound diagnosis apparatus according to claim 1, wherein, during motion of the ultrasound probe, the processing circuitry corrects a position of the ultrasound probe, on a basis of at least one selected from among: skeleton information of an examined subject, body surface information of the examined subject, scan angle information of the ultrasound probe, contact pressure information of the ultrasound probe, cardiac phase information of the examined subject, and respiratory phase information of the examined subject.

9. An imaging method comprising:
three-dimensionally performing a scan by using a robot arm capable of moving and rotating an ultrasound probe; and
evaluating ultrasound data obtained from the scan so as to judge whether or not it is necessary to perform a re-scan using the robot arm.

10. A computer program product having a computer-readable recording medium including a plurality of computer-executable instructions, wherein the plurality of instructions cause a computer to execute:
three-dimensionally performing a scan by using a robot arm capable of moving and rotating an ultrasound probe; and
evaluating ultrasound data obtained from the scan so as to judge whether or not it is necessary to perform a re-scan using the robot arm.
